(19) United States
(12) Patent Application Publication
(10) Pub. No.: US 2010/0022472 A1
(43) Pub. Date: Jan. 28, 2010

(54) LIQUID, EYE-INSTILLABLE PREPARATIONS COMPRISING SODIUM HYALURONATE

(75) Inventor: David James Lloyd, Surrey (GB)

Correspondence Address: CALFEE HALTER & GRISWOLD, LLP, 800 SUPERIOR AVENUE, SUITE 1400, CLEVELAND, OH, 44114, US

(73) Assignee: RENAISSANCE HEALTHCARE (EUROPE) LIMITED, Surrey (GB)

(21) Appl. No.: 12/573,536

(22) Filed: Oct. 5, 2009

Related U.S. Application Data
(63) Continuation of application No. 10/449,548, filed on May 30, 2003, now abandoned.

(30) Foreign Application Priority Data
Dec. 20, 2001 (GB) 0130603.4
Dec. 20, 2002 (GB) PCT/GB02/05834

(51) Int. Cl. A61K 31/728; A61P 27/02

(52) U.S. Cl. 514/54

(57) ABSTRACT

A liquid, eye-instillable preparation comprises a viscosity-enhancing agent comprised of one or both of sodium hyaluronate and chondroitin sulphate, a preservative comprised of polyhexanide, and one or more carriers in which the agent and the preservative are dispersed.

This invention relates to a liquid, eye-instillable preparation.

Sodium hyaluronate (hyaluronic acid sodium salt) (SH) is a viscoelastic mucopolysaccharide found naturally in the skin, synovial fluid, and vitreous humour of the eye to name a few. A major physical property is its ability to respond to an applied force, i.e. in the passive state it forms a gel but when a force is applied (active state) it changes to a liquid, i.e. has non-Newtonian rheological properties. It is used in various applications in medicine, e.g in the treatment of osteoarthritis in joints such as the knee it replaces the synovial fluid which has been lost as a result of the disease; this is called viscosupplementation. It is also used in surgical procedures such as cataract operations, where the SH protects the inner layers of the eye (e.g. the endothelium) and also allows the surgeon to manipulate instruments without damaging the eye; this is called viscoprotection. There are several SH products on the market today that are used in viscosupplementation and viscoprotection.

The physical properties of SH mean that it is used in formulating eye drop preparations. Not only is SH a natural component of tears, and thus very biocompatible, but also it is an excellent lubricating ingredient for treating ocular surface disease such as dry eye syndrome where dry eye syndrome ranges from mere sensation, e.g. itchy, scratchy, gritty, tired, red, burning and watery, to pathological dry eye, e.g. Sjogrens syndrome, keratoconjunctivitis sicca, and Stevens Johnson syndrome. SH has the ability to retain vast quantities of water and so it imparts a very comfortable sensation when instilled into the eye and also has excellent retention properties, i.e. it remains a lot longer in the eye compared with other traditional eye drop products. A number of preservative-free SH-containing, eye drop preparations, both single-dose and multi-dose, are on the global market. However, such eye drops are relatively expensive and this unfortunately precludes certain patients, for example those with little disposable income, from purchasing such products. These patients are inclined to buy multi-dose eye drops containing inferior lubricants and a preservative that is likely to cause irritation and exacerbate the condition which they are trying to alleviate.

There are several eye drop preparations on the global market that do contain preservatives, in the main benzalkonium chloride, as this affords good anti-microbial activity; however, it can be toxic to the epithelial layer of the eye.

Polyhexanide (polyhexamethylene biguanide or polyaminopropyl biguanide) (PHMB) is a polymeric biguanide anti-microbial agent used for disinfecting, inter alia, swimming pools, dairy pipes and beer lines. PHMB is also used in contact lens care products because of its low toxicity and excellent anti-microbial activity.

However, PHMB in chemical terms is a cationic molecule (net +ve charge), whereas SH is an anionic molecule (net −ve charge). It is known by chemists skilled in the art of formulating products that when +ve charged molecules and −ve charged molecules are mixed together they are incompatible, i.e. their specific individual physicochemical properties disappear or are severely impaired.

In the case of SH and PHMB one could postulate that, if they were to be mixed together, the antimicrobial activity of PHMB would be inactivated and in the case of SH the viscosity would be considerably reduced.

According to the present invention, there is provided a liquid, eye-instillable preparation comprising a viscosity-enhancing agent comprised of one or both of sodium hyaluronate and chondroitin sulphate, a preservative comprised of polyhexanide, and one or more carriers in which the agent and the preservative are dispersed.

Owing to the invention, it is possible to improve liquid, eye-instillable preparations, for example as regards their efficacy and/or their cost effectiveness.

Advantageously, the preparation does not contain any non-ionic or amphoteric substances.

The preparation may be administered as an eye drop or a lotion in the form of an approximately isotonic aqueous solution.

The agent comprising SH and/or chondroitin sulphate, is advantageously in a range of 0.01 to 2.0% w/v, preferably 0.05 to 0.5 w/v, of the solution.

In the case of SH, the molecular weight is preferably in the range of 400,000 to 3 million Daltons, and may be a mixture of molecular weights, with the SH characterised by narrow molecular weight distribution bands with low levels of inflammatory contaminants from the manufacturing and purification process and where the concentration of SH is 0.05 to 0.4% w/v.

The preservative, comprised of PHMB, is advantageously in a range of 0.1 to 5.0 ppm, preferably 0.5 to 2.0 ppm, of the solution.

In the case of mixing SH and PHMB it was surprising to observe that, when the two substances were mixed, there was no sign of complexation e.g. cloudiness, which would have indicated physical incompatibility. Complexation would also decrease the viscosity of SH and possibly render the antimicrobial activity of PHMB ineffective. It was thus even more surprising that, when the PHMB-preserved SH-solution was challenge-tested against the four marker micro-organisms recommended in the European Pharmacopoeia, sufficient antimicrobial activity was demonstrated. It would seem that the active antimicrobial groups on the PHMB molecule were still bioavailable. In addition the viscosity of the SH-PHMB solution was unimpaired.

A preferred embodiment of the invention is a sterile, buffered, slightly hypotonic, preserved eye drop presented in multi-dose units, e.g. of 10 ml. It contains the active principal sodium hyaluronate PhEur (SH), 0.14% w/v and the preservative PHMB, 2 ppm. SH-PHMB is used as an ocular lubricant where sensation of dryness as well as burning and ocular fatigue, due to dust, smoke, dry heat, air conditioning, extended computer use, contact lens wear, etc., is indicated.

The preferred embodiment also includes conventional buffering and chelating agents and one or more conventional carriers, e.g. water, in conventional proportions.

The SH is obtained by biological fermentation. With current regulatory concerns over materials derived from animal origin it is reassuring that the source of the SH is from biological fermentation and not from rooster combs, which was, until very recently, the main commercial source of SH. This microbiological source of SH precludes concerns over the presence of avian proteins. SH is a polysaccharide made up of linear repeating units of a disaccharide (10 to 10,000 repeats) made up of D-glucuronic acid and D-N-acetylglucosamine linked together by alternating beta-1,4 and beta 1,3 glycosidic bonds. SH as a biological substance is described in Merck Index, 11^{th} edition (Merck Index No.: 12, 4793) and is monographed in the European Pharmacopoeia, 3^{rd} Edition, Supplement 2000, pages 1190-1193 (Appendix 3). The CAS No. for sodium hyaluronate is 9067-32-7.

SH, contrary to earlier belief, is ambiphilic (both hydrophobic {axial hydrogens} and hydrophilic regions). The eye drop formulation uses SH of a molecular weight in the range of 1 to 3 million Daltons.

SH is found naturally in the pre-corneal tear fluid of the eye and its concentration is dependent on the physiological condition of the surrounding tissue. Thus the immediate benefit, with respect to physiological compatibility, of using a natural component of tear fluid in an eye drop formulation becomes apparent.

The other physical benefits that SH can offer in eye drop preparations are summarised below. All of these benefits support the use of SH in the treatment of patients with dryness sensation whether it is related to environmental conditions or contact lens wear, for example:

Lubrication:
The “slippery nature and feel” of the hyaluronans make them ideal candidates for providing lubrication (comfort) to compromised mucous layers.

Water Retention:
1 gram of SH has the ability to retain up to 6 litres of water. Thus it provides an aqueous reservoir which will “hydrate” the surrounding medium.

Mucin Synergy (Bio-Adhesiveness or Muco-Adhesiveness):
SH has the ability to bind or interact with the mucin layer of the pre-ocular tear film, thus contributing to increased ocular residence times.

Increased Residence Times:
SH has significantly improved residence times in the eye over other eye lubricants, e.g. polyvinyl alcohol and hydroxypropylmethylcellulose.

Increased Tear Thickness:
SH by virtue of its viscosity and general physical behavior will increase the thickness of the pre-corneal tear film.

Increased TBUT (Tear Break-Up Time):
As a consequence of its muco-adhesiveness and prolonged residence times SH will stabilise the pre-corneal tear film and so improve the TBUT.

Drug Delivery:
SH will act as a carrier for compatible drugs and due to muco-adherent properties will allow prolonged contact times on the ocular surface.

Theologically Responsive:
Since SH is viscoelastic and thus exhibits non-Newtonian behaviour, the viscosity will respond to shear such that it will change from a gel to a sol. Since the tear film also responds to shear from blinking, then SH will mimic that action. SH offers less resistance to eyelid movement over the globe.

Corneal Wound Healing
SH has been implicated in wound healing and this has been shown to be evident on the cornea.

It is the properties mentioned hereinbefore that specifically place the present SR-containing eye drop formulation above more traditional lubricant eye drop preparations, e.g. from those containing merely saline to those containing ingredients such as polyvinyl alcohol, hydroxpropylmethylcellulose, carboxymethylcellulose, polyvinyl pyrrolidone and carbomer gels.

This present SH-containing eye drop exhibits non-Newtonian properties and so the apparent viscosity decreases as shear stress (shear thinning) increases. It is therefore affected by blinking and rapid eye movement, resulting in thinning and so offering less resistance to eyelid movement over the globe.

One of the primary pre-requisites of a product for the treatment of sensation of dry eye is the duration of the active ingredient in the eye. SH has the necessary properties to accommodate this pre-requisite.

1-14. (canceled)

15. A liquid, eye-instillable preparation comprising a viscosity-enhancing agent comprised of one or both of sodium hyaluronate and chondroitin sulphate, a preservative comprised of polyhexanide, and one or more carriers in which the agent and the preservative are dispersed, wherein said agent is present in a range of 0.01 to 2.0% w/v of the dispersion.

16. A preparation according to claim 15, wherein the preparation is essentially free of non-ionic substances.

17. A preparation according to claim 15, wherein the preparation is essentially free of amphoteric substances.

18. A preparation according to claim 15, and in the form of an approximately isotonic aqueous solution.

19. A preparation according to claim 15, wherein said agent is present in a range of about 0.05 to 0.5% w/v of the dispersion.

20. A preparation according to claim 19, wherein said preparation contains about 0.14% w/v sodium hyaluronate and about 2 ppm polyhexanide.

21. A preparation according to claim 15, wherein said preparation contains sodium hyaluronate of molecular weight in a range of 400,000 to 3 million Daltons.

22. A preparation according to claim 21, wherein said molecular weight is in a range of 1 to 3 million Daltons.

23. A preparation according to claim 15, wherein said agent is sodium hyaluronate and has been obtained by biological fermentation.

24. A preparation according to claim 15, wherein said polyhexanide is in a range of 0.1 to 5.0 ppm of the dispersion.

25. A preparation according to claim 24, wherein said polyhexanide is in a range of 0.5 to 2.0 ppm of the dispersion.

26. A liquid, eye-instillable preparation according to claim 15, wherein the preparation comprises
a viscosity-enhancing agent comprising one or both of sodium hyaluronate and chondroitin sulphate in an amount of about 0.01 to 2.0% w/v of the preparation,
a preservative comprising polyhexanide in an amount of about 0.1 to 5 ppm of the preparation, and
one or more carriers in which the agent and the preservative are dispersed,
wherein the preparation is essentially free of a non-ionic substance.

27. The preparation of claim 26, wherein the preparation contains about 0.05 to 0.5% w/v viscosity-enhancing agent and about 0.5 to 2 ppm polyhexanide.

28. The preparation of claim 27, wherein the preparation contains about 0.05 to 0.5% sodium hyaluronate having a molecular weight of about 400,000 to 3 million Daltons.

29. A multi-dose eye drop unit containing a liquid, eye-instillable preparation in the form of a sterile, buffered, slightly hypotonic, eye drop solution and comprising a viscosity-enhancing agent comprised of one or both of sodium hyaluronate and chondroitin sulphate, a preservative comprised of polyhexanide, and one or more carriers in which the agent and the preservative are dispersed, wherein said agent is in a range of 0.01 to 2.0% w/v of the dispersion.

30. A unit according to claim 29, wherein the preparation is essentially free of non-ionic substances.

31. A unit according to claim 29, wherein the preparation is essentially free of amphoteric substances.

32. A unit according to claim 29, wherein said preparation is in the form of an approximately isotonic aqueous solution.

33. A unit according to claim 29, wherein said agent is sodium hyaluronate of molecular weight in a range of 400,000 to 3 million Daltons.

34. A multi-dose eye drop unit according to claim 29, wherein the preparation comprises
a viscosity-enhancing agent comprising one or both of sodium hyaluronate and chondroitin sulphate in an amount of about 0.01 to 2.0% w/v of the preparation,
a preservative comprising polyhexanide in an amount of about 0.1 to 5 ppm of the preparation, and
one or more carriers in which the agent and the preservative are dispersed,
wherein the preparation is essentially free of a non-ionic substance.

35. The multi-dose eye drop of claim 34, wherein the preparation contains about 0.05 to 0.5% w/v viscosity-enhancing agent and about 0.5 to 2 ppm polyhexanide.

36. The multi-dose eye drop of claim 35, wherein the preparation contains about 0.05 to 0.5% sodium hyaluronate having a molecular weight of about 400,000 to 3 million Daltons.
